(19) United States
(12) Patent Application Publication
(10) Pub. No.: US 2017/0071784 A1
(43) Pub. Date: Mar. 16, 2017

(54) High efficiency apparatus for selectively heating and/or cooling therapeutic gel packs by conduction

(71) Applicant: William I. Brobeck, Moraga, CA (US)

(72) Inventor: William I. Brobeck, Moraga, CA (US)

(21) Appl. No.: 14/756,570

(22) Filed: Sep. 16, 2015

(51) Int. Cl. A61F 7/02; H05B 3/26; H05B 3/28; F25B 13/00

(52) U.S. Cl. CPC A61F 7/0241; F25B 13/00; H05B 3/26; H05B 3/28; A61F 2007/0094

(57) ABSTRACT

A bedside, compact system is provided which either selectively heats or cools a gel pack to assist a user in dealing with pain. The system heats and cools by conduction and operates quickly to provide a heated or cooled gel pack within minutes. The user can operate the system with getting out of bed or other resting place.

### BACKGROUND

The present invention pertains generally to the treatment of pain with “hot packs” and/or “cold packs,” also commonly referred to as thermal gel packs, therapeutic gel packs, or simply gel packs. A typical gel pack can be used as either a hot or cold pack. More particularly, the most preferred embodiment of the invention is a small, bedside unit that can quickly and efficiently either heat or cool a therapeutic thermal gel pack for the user; the user does not have to get out of bed or other resting place.

Many types of therapeutic gel packs are available to millions of users who either have chronic or temporary pain. The user typically either has to get out of bed (or other resting place) or has to rely on another person to provide either a hot or cold pack. The number of persons with chronic pain in the United States is more than 100,000,000 and continues to increase. More and more persons with chronic pain live alone and remain in bed or at rest for extended periods each day. Athletes with temporary pain can benefit from the present invention since the gel pack is heated or cooled very quickly.

There is a huge and rising demand for a system that includes a small bedside unit that provides either a hot or cold pack for a user whereby the user can repeatedly obtain either a hot or cold pack quickly without getting out of bed or other resting place. The present invention provides such a system.

### BRIEF SUMMARY OF INVENTION

The present invention is a small unit which provides heating and/or cooling by conduction, and has an internal chamber of less than 0.5 cubic foot. At least one wall of the chamber has a high thermal conductivity, such as aluminum, copper or stainless steel. The therapeutic gel pack is brought into direct physical contact with the thermally conductive wall and is either heated or cooled to a desired temperature rapidly by conduction. The thermal conductivity of aluminum is roughly 8,000 times greater than the thermal conductivity of air; copper is about 16,000 times more conductive than air. Stainless steel has a thermal conductivity about 600 times greater than air. The present invention either heats or cools the therapeutic gel pad by conduction. It is inherently capable of heating or cooling the pads much more quickly than standard air chambers used in common refrigerators and heaters.

The present invention is preferably thermostatically controlled and is capable of heating or cooling the thermal pad to any desired temperature.

The present invention is capable of remote actuation by a cell phone or other similar remote means.

A primary object of the invention is to provide a thermal care system wherein a relatively small bedside or chair-side unit either heats or cools a therapeutic gel pack to a desired temperature by conduction.

A further object of the invention is to provide a system wherein a user can obtain a heated or cooled gel pack quickly without getting out of bed, or other resting place, and without any help from another person.

Further objects and advantages of the invention will become apparent from the following description and drawings.

### BRIEF DESCRIPTION OF DRAWINGS

FIG. 1 is a schematic representation of the system wherein a gel pack is in contact with a thermally conductive wall and wherein the system is either heating or cooling the gel pack;

FIG. 2 is a schematic showing the gel pack separated from the thermally conductive wall;

FIG. 3 is a schematic showing the gel pack being slid outwardly from the heating or cooling chamber to be accessible by a user;

FIG. 4 is a front perspective view of the device;

FIG. 5 is a rear perspective view of the device; and

FIG. 6 is a schematic showing a second embodiment of the invention, which resembles a waffle iron.

FIG. 7 is a perspective view of the second embodiment shown in FIG. 6; and

FIG. 8 is a graph showing temperature versus time for cooling a gel pack.

### DETAILED DESCRIPTION OF THE DRAWINGS

The present invention is designed to bring hot and cold gel pack therapy convenience to the user. Instead of having to walk back and forth to the kitchen, the user can place the device near his/her bed, couch or chair and have easy access to therapy gel packs. The energy efficient system will either heat or cool a gel pack to optimum temperature in approximately 20 minutes or less allowing the user to remain as comfortable as possible thereby minimizing the possibility of any re-injury. The system in one embodiment of the invention can be outfitted with one or more trays allowing the user to apply cold and heated gel packs at the same time to different parts of the body or to alternate between hot and cold. The system with multiple trays could also be used in a physical therapy office making reusable gel packs accessible because of the quicker heating or cooling time required.

The invention can be used by amateur and professional athletes, since the system heats and/or cools the gel packs so quickly.

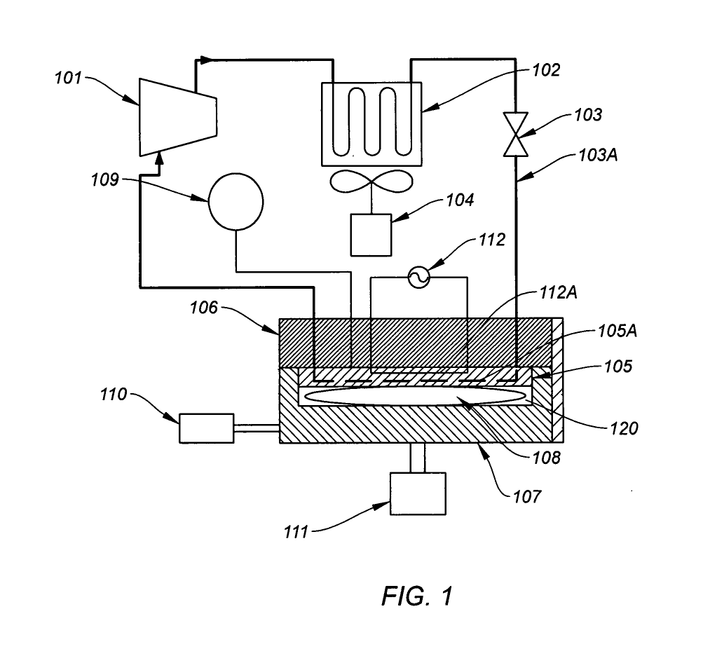

FIGS. 1-5 illustrate an embodiment using a single tray.

As shown in FIG. 1 a refrigeration compressor 101, a condenser 102, an expansion valve 103 and a condenser fan 104, all as known in the art, are provided. A highly conductive plate 105 forms a wall of chamber 120. Plate 105 may be made preferably of copper, or aluminum, for example. An evaporator cold plate 105a is connected to refrigerant line 103a and is thermally connected to or imbedded in conductive plate 105. Other refrigeration systems known in the art may be used.

Also shown in FIG. 1 is an electronic power supply 112 connected to heating coils 112a embedded in or thermally connected to conductive plate 105. Power supply 112 is controlled by a combined microprocessor and thermostat 109.

An insulation layer 106 is mounted adjacent conductive plate 105.

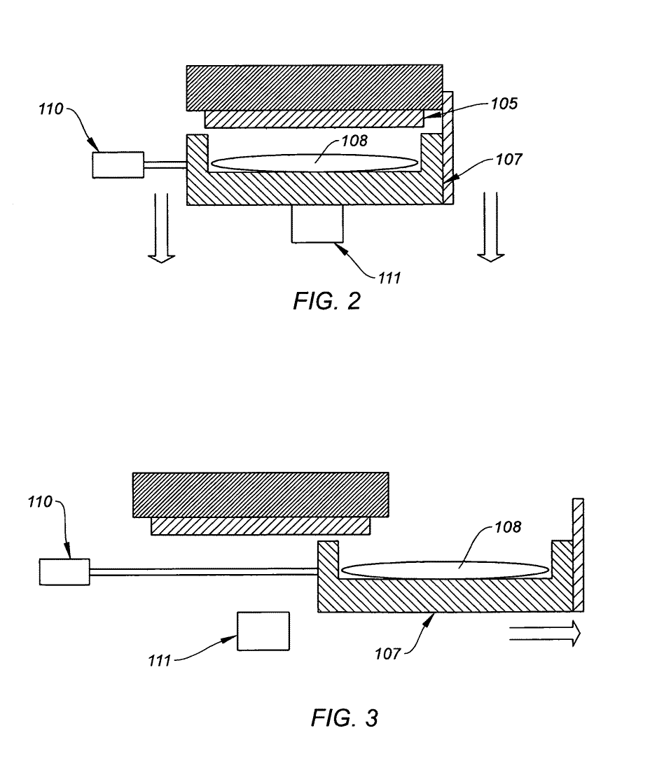

A sealing actuator 111 is removably connected to tray 107 and moves tray 107 vertically as shown in FIG. 2 to a second, closed position shown in FIG. 1 wherein the gel pack 108 is in thermal and physical contact with conductive plate 105.

A horizontal actuator 110 is connected to tray 107 and moves tray 107 from a first, open or extended position shown in FIG. 3 to a second, retracted position shown in FIG. 2.

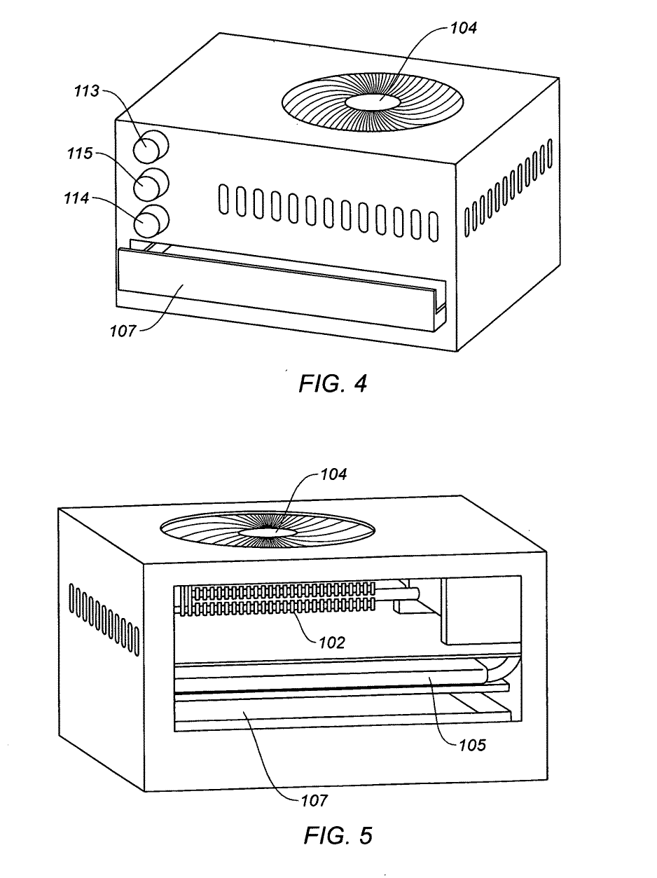

FIGS. 4 and 5 are front and rear exterior, perspective views of the single tray unit shown in FIGS. 1-3. Reference numerals in FIGS. 4 and 5 correspond with these in FIGS. 1-3. Multiple units can be utilized to allow the user to heat one gel pack simultaneously with cooling a second gel pack.

To operate the device, the user presses the Open/Close tray pushbutton 114 (FIG. 4), and the tray 107 is opened, as shown in FIG. 3. The user places gel pack 108 in tray 107 and presses pushbutton 114 again. Tray 107 is retracted by actuator 110 to the position shown in FIG. 2.

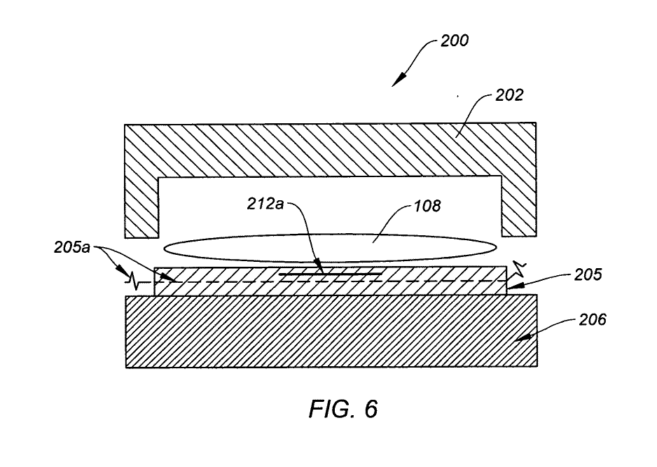

After the tray door is closed, the sealing actuator 111 pushes up the tray 107 against the thermally conductive surface of plate 105. Now the gel pack 108 is in direct physical contact with the plate 105 so heat transfer is by conduction which is substantially more efficient than convection. Depending on which mode the user selects with Warm/Cold pushbutton 115, it starts to warm or cool. For cooling, the compressor 101 starts and controller 109 monitors the gel pack 108 temperature. As the temperature of the gel pack transitions through its phase change, as shown in FIG. 6 the temperature will level off. After the gel pack has completed its phase change the temperature level will begin to drop again. That's when the compressor will stop and the device will signal “Relief is Ready” 113. For warming the microprocessor temperature controller 109 will turn on the power supply for the heating in 112a. When the gel pack temperature has reached a warm but still safe temperature, the “Relief is Ready” 113 light will blink.

Today a gel pack sold at CVS recommends leaving the gel pack in the freezer for a minimum of 1 hour before use. The present system is designed to press the room temperature gel pack 108 against its refrigerated cold plate 105 so the heat transfer is by conduction which is significantly more efficient than any standard refrigerator/freezer that uses natural or forced air convection as is means for heat transfer. Therefore the present refrigeration system is dedicated to cool a gel pack 108 by the most efficient means possible.

The present system is designed to cool gel packs of different chemical compositions to optimum temperature automatically. The microprocessor 109 will monitor the gel pack 108 temperature. In particular it will recognize when the gel pack goes through its phase change (FIG. 6). When the gel pack's phase change is complete and its temperature begins to fall again, the system will turn on the “Relief is Ready” light 113. This means that the present system can be used with virtually any special gel pack that will not be too cold and can be directly applied to the skin.

The system may also be used to warm a gel pack. The user pushes the Warm/Cold pushbutton 115 until the light turns from Blue (cold) to Red (warm). The user places the gel pack 108 on the tray 107 and pushes button 114. The gel pack tray will be pushed up against the now warming plate and be heated to the maximum allowable temperature so as not to burn the skin. The same microprocessor temperature control will illuminate the “Relief is Ready” light (13) when the gel pack is warm enough.

The system also has a shutdown monitor. If the unit has not be accessed for more than 4 hours (this is adjustable), it will shut down.

The system will have internet access with the user's smartphone through an application allowing the user to begin heating or cooling of a gel pack remotely. For instance, the user may remotely start the system before driving home after a game of tennis. When the user arrives at home, the gel pack will be ready for use.

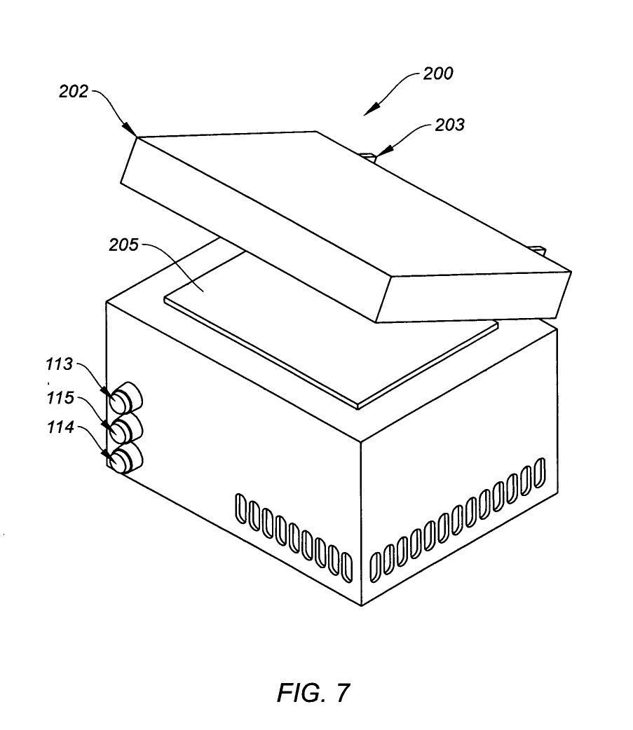

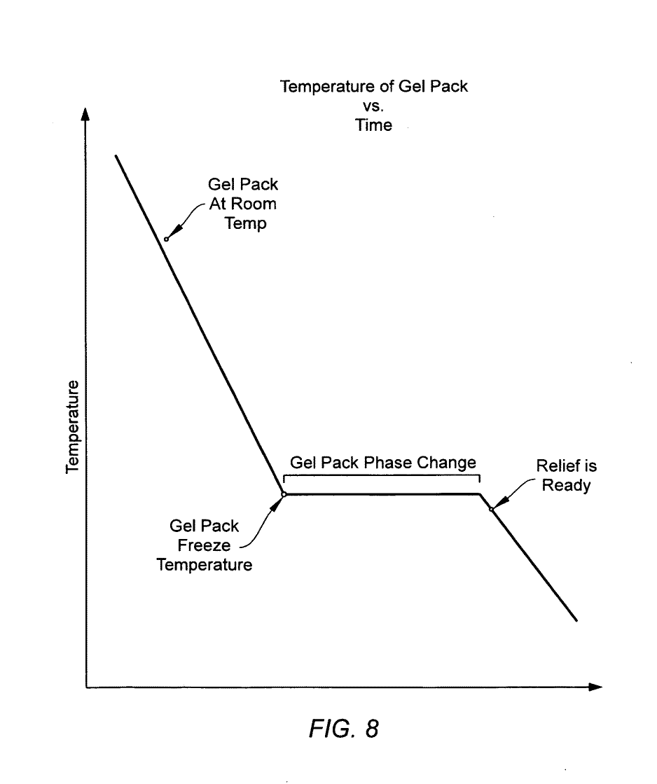

FIGS. 6 and 7 illustrate a second embodiment 200 of the invention, which resembles a waffle iron. A highly thermally conductive wall 205 is supporting a gel pack 208. Wall 205 is supported by an insulating base 206. A movable lid 201 may be hinged by hinges 203 (FIG. 7) or may be provided with a handle (not shown). The cooling refrigerant line 205a is shown as embedded in conductive plate 205, along with heating coil 212a. Connection of the cooling line 205a and heating coil 212a would be similar to those shown in FIG. 1, and are not shown in FIG. 6 or 7 for brevity and clarity.

FIG. 7 shown the housing 250 and hinged lid 201. Control buttons 113, 114 and 115 are the same as those shown in FIG. 4.

The foregoing description of the invention has been presented for purposes of illustration and description and is not intended to be exhaustive or to limit the invention to the precise form disclosed. Modifications and variations are possible in light of the above teaching. The embodiments were chosen and described to best explain the principles of the invention and its practical application to thereby enable others skilled in the art to best use the invention in various embodiments suited to the particular use contemplated.

1. Apparatus for selectively heating or cooling a therapeutic gel pack to a predetermined temperature comprising
a chamber
a therapeutic gel pack removably positioned in said chamber
a thermally conductive surface forming a wall of said chamber
means for causing said therapeutic gel pack to move into thermal and physical contact with said thermally conductive surface
means for selectively either heating or cooling said thermally conductive surface, whereby said therapeutic gel pack is either heated or cooled by conduction to said predetermined temperature.

2. The apparatus of claim 1 further comprising a slidable tray for moving said therapeutic gel pack into and out of said chamber.

3. The apparatus of claim 2 further comprising means for remotely actuating said apparatus.

4. The apparatus of claim 1 further comprising:
a heating unit,
a refrigeration unit,
means for connecting either said heating unit or said refrigeration unit to said thermally conductive surface.

5. The apparatus of claim 1 further comprising thermostatic means for determining the final temperature to which said therapeutic gel pack is either heated or cooled.
